(19) United States
(12) Patent Application Publication
(10) Pub. No.: US 2025/0186726 A1
(43) Pub. Date: Jun. 12, 2025

(54) DEVICE FOR REPLACING THE ORAL TRACHEAL INTUBATION WITH NASAL TRACHEAL INTUBATION

(71) Applicant: First Affiliated Hospital of Kunming Medical University, Kunming (CN)

(72) Inventors: Qian Wu, Kunming (CN); Lin Zhu, Kunming (CN); Shujuan Dai, Kunming (CN)

(21) Appl. No.: 18/431,929

(22) Filed: Feb. 3, 2024

(30) Foreign Application Priority Data
Dec. 9, 2023 (CN) 202311683228.0

(51) Int. Cl. A61M 16/04

(52) U.S. Cl. CPC A61M 16/0461; A61M 16/0488

(57) ABSTRACT

The invention discloses a device for replacing the oral tracheal intubation with nasal tracheal intubation, comprising a semicircular tube and a guide tube, the side of the semicircular tube being provided with an opening for placement of the guide tube or tracheal intubation; the diameter of the guide tube is smaller than that of the semicircular tube, and magnetic portions paired with each other are provided on the inner wall of the semicircular tube as well as on the side wall of the guide tube near the end position. The invention can effectively assist in replacing the oral tracheal intubation with nasal tracheal intubation, reduce the multiple risks that may exist in the process of replacing intubation, improve the patient's respiratory support method, and improve the prognosis.

### 1. TECHNICAL FIELD

The invention relates to the technical field of medical equipment, in particular to a device for replacing the oral tracheal intubation with nasal tracheal intubation.

### 2. BACKGROUND ART

Tracheal intubation is an important measure to ensure that critically ill patients have an unobstructed respiratory tract, improve ventilation, and maintain oxygenation; it includes two approaches, namely oral tracheal intubation and nasal tracheal intubation.

Oral tracheal intubation is relatively simple and rapid, with short treatment time and high success rate of intubation, and is often used in the rescue of patients with acute and critical illnesses, which can rapidly improve the ventilation of patients. However, there are obvious shortcomings in the subsequent long-term treatment:

1. Adverse effects on the physiological functions of the patient's oral cavity and its related structures; on the one hand, prolonged retention of oral tracheal intubation may cause complications such as sore throat, hoarseness, bloody sputum, damage to the maxillary mucous membrane, swelling of the tongue under pressure, and obstruction of oral cavity venous return; on the other hand, keeping the tube in the mouth for a long time passively may lead to transient or permanent dislocation of the mandibular joint of the patient.

2. Adverse effects on patients' oral hygiene. The fixation of oral tracheal intubation is affected by many factors, including dental integrity, occlusal function, cough reflex, and state of consciousness. For example, in the case of elderly patients, the physiological loss of teeth, unstable dental bed and weakened occlusal ability make the oral tracheal intubation lose a relatively stable fixed fulcrum and prone to be dislodged. And then, such as young patients, their cough reflex is strong, occlusal ability is strong, but the intubation tolerance is poor, tracheal intubation is often coughed out due to restlessness, resulting in interruption of air delivery; or bite the intubation to cause the tracheal intubation collapsed, resulting in difficulties in air delivery.

3. Tracheal intubation is highly mobile and poorly tolerated. Tracheal intubation is delivered through the oral cavity, which can move left and right in the oral cavity, and also move up and down with swallowing, therefore, it is irritating to the throat and the posterior pharyngeal wall, causing regurgitation, nausea and other discomforts, and therefore, it is poorly tolerated.

Therefore, in clinical work, the retention time of tracheal intubation is mostly from 3 to 7 days, and the longest is not more than 14 days. In patients who are predicted to require prolonged airway access during the course of their disease, early consideration should be given to switching to noninvasive nasal tracheal intubation or invasive temporary tracheotomy.

Nasal tracheal intubation has the advantages of easy to tolerate, easy to fixate, easy to care for, and longer retention time, etc.: 1. Unlike oral tracheal intubation, nasal tracheal intubation is entered from the nasal cavity, and thus has relatively less mobility, is easy to fixate, and reduces the risk of tube detachment, as well as is more convenient to the patient's oral care as well as feeding through the mouth; 2. nasal tracheal intubation is less stimulating to the throat, and the occurrence of vomiting reflex is low, which is easier for patients to tolerate, and is helpful to reduce the use of sedative and analgesic drugs in clinical practice. 3. The time of relying on mechanical ventilation of patients with early nasal tracheal intubation is significantly shorter than that of patients with oral tracheal intubation, and even some patients have avoided going through tracheotomy; and family members of the patients and some awake patients have higher acceptance for nasal tracheal intubation.

Therefore, critically ill patients whose airways are opened by oral tracheal intubation in emergency situations need to be replaced with nasal tracheal intubation after evaluation by a physician. Because the oral and nasal routes are not connected, the current clinical practice requires completely removing the oral tracheal intubation, followed by blind insertion, laryngoscope, fiberscope, light stick, or ultrasound assistance and other methods for reintubation through the nose. This process has a high degree of difficulty and risk: 1. From the physiological point of view, the airway mucosa that has already undergone oral tracheal intubation has a certain degree of hyperaemia and edema, and the anatomical structure may not be clear even with the assistance of visual laryngoscopy; 2. When the oral tracheal intubation is removed from a critically ill patient, there is a large amount of phlegm or airway secretion coming out from under the glottis, which further decreases the visual field visibility near the glottis, 3. When the oral tracheal intubation is removed, it is tantamount to temporary and complete disconnection of respiratory assisted support, and the patient may experience a rapid decline in oxygen saturation or oxygenation; 4. The use of adequate amounts of anesthetics or muscle relaxants inhibits the patient's autonomous respiration on the one hand, and lowers body temperature and blood pressure on the other, which may result in the inability to maintain vital signs in life-threatening situations.

Therefore, tracheal intubated patients need a quick and easy-to-use assistance device that can assist in tube replacement, realize “seamless connection” throughout the process of tube replacement, access respiratory assistance devices at any time, avoid damage to airway due to repeated intubation, and achieve the goal of increasing the success rate of tube replacement, decreasing the risk of tube replacement, and ultimately improving the prognosis of patients.

### 3. SUMMARY OF THE INVENTION

The invention aims to overcome said technical problems and provides a device for replacing the oral tracheal intubation with nasal tracheal intubation. In order to realize said purpose, the invention adopts said technical scheme:
a device for replacing the oral tracheal intubation with nasal tracheal intubation, comprising a semicircular tube and a guide tube, the side of the semicircular tube being provided with an opening for placement of the guide tube or tracheal intubation; the diameter of the guide tube is smaller than that of the semicircular tube, and magnetic portions paired with each other are provided on the inner wall of the semicircular tube as well as on the side wall of the guide tube near the end position.

As an improvement, said opening extend to two ends of the semicircular tube so that the semicircular tube is U-shaped without external force.

As an improvement, said guide tube is provided with a scale showing the depth of insertion.

As an improvement, said guide tube scale is shown as 5-65 cm and said guide tube has a length of 65-75 cm.

As an improvement, said magnetic portions are provided on the inner wall of the semicircular tube near the middle position.

The advantages of the invention are as follows:

The invention adopts semicircular tube cooperating with the guiding tube to provide assistance guidance for tracheal intubation, which can effectively reduce the difficulty of tube changing and improve the success rate of tube changing.

The invention can minimize the risk of sudden changes in the patient's vital signs and endangerment of life caused by having to temporarily disengage from respiratory assistance and use anesthetics and muscle relaxant during the tube changing process of “oral tracheal intubation-nasal tracheal intubation”; and at the same time, it can also significantly improve the one-time success rate of tube change, avoiding repeated intubation and aggravating airway damage. At the same time, this device is simple to operate and easy to learn, and can be popularized to the grassroots hospitals, thus realizing its important clinical application value.

### 4. BRIEF DESCRIPTION OF ACCOMPANY DRAWINGS

FIG. 1 is the structural diagram of the semicircular tube of embodiment 1.

FIG. 2 is the structural diagram of the guide tube of embodiment 1.

FIG. 3 is the diameter contrast diagram of the semicircular tube, guide tube, and oral tracheal intubation of embodiment 1.

FIG. 4 is the anatomical diagram of the position of the mouth, nose and airway.

FIG. 5 to FIG. 15 are the operational flowchart illustrating replacing the oral tracheal intubation with nasal tracheal intubation of embodiment 1.

FIG. 16 is the structural diagram illustrating when the semicircular tube is nested with the oral tracheal intubation.

FIG. 17 is the structural diagram illustrating when the guide tube is nested with the nasal tracheal intubation.

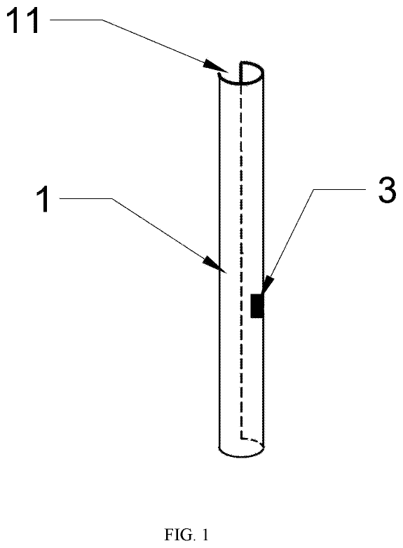

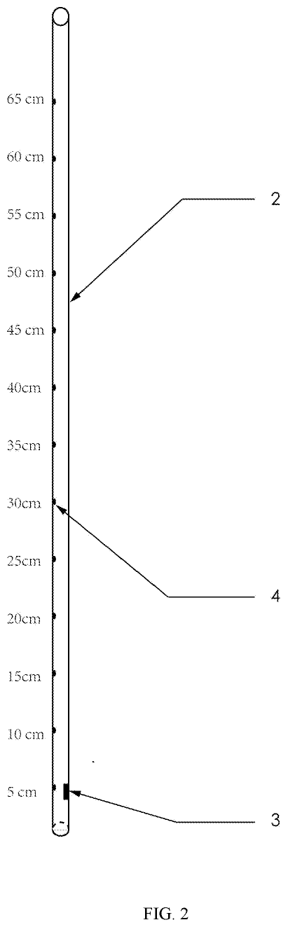

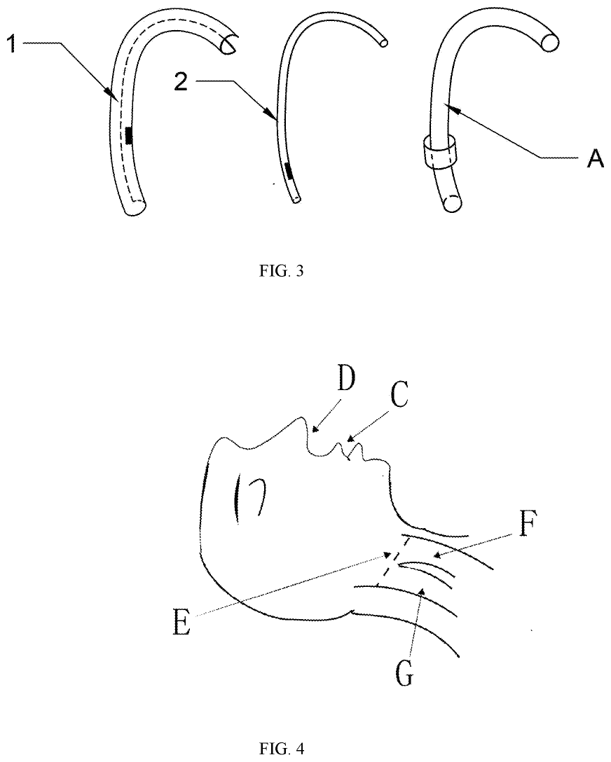

As shown in the accompanying drawings: 1—semicircular tube, 11—opening, 2—guide tube, 3—magnetic portion, 4—scale.

A—oral tracheal intubation, B—nasal tracheal intubation, C—oral cavity, D—nasal cavity, E—glottis, F—airway, G—esophagus.

### 5. SPECIFIC EMBODIMENT OF THE INVENTION

In order to make the objects, technical solutions and advantages of the embodiments of the present invention clearer, the technical solutions in the embodiments of the invention will be clearly and completely described below with reference to the drawings in the embodiments of the invention, and it is obvious that the described embodiments are some, but not all, embodiments of the invention. The components of embodiments of the invention generally described and illustrated in the figures herein may be arranged and designed in a wide variety of different configurations.

In the description of the embodiments of the invention, it should be noted that, if the terms “center”, “upper”, “lower”, “left”, “right”, “vertical”, “horizontal”, “inner”, “outer”, etc. indicate orientations or positional relationships based on the orientations or positional relationships shown in the drawings or the orientations or positional relationships that the products of the invention are usually placed in when used, the orientations or positional relationships are only used for convenience of describing the invention and simplifying the description, but the terms do not indicate or imply that the devices or elements indicated must have specific orientations, be constructed in specific orientations, and operate, and therefore, should not be construed as limiting the invention. Furthermore, the terms “first”, “second”, “third” and the like are used solely to distinguish one from another and are not to be construed as indicating or implying relative importance.

Furthermore, the terms “horizontal”, “vertical”, “overhang” and the like do not require that the components be absolutely horizontal or overhang, but may be slightly inclined. For example, “horizontal” merely means that the direction is more horizontal than “vertical” and does not mean that the structure must be perfectly horizontal, but may be slightly inclined.

In the description of the embodiments of the invention, “a plurality” represents at least 2.

In the description of the embodiments of the invention, it should be further noted that unless otherwise explicitly stated or limited, the terms “arranged”, “mounted”, and “connected” should be interpreted broadly, and may be, for example, fixedly connected, detachably connected, or integrally connected; can be mechanically or electrically connected; they may be connected directly or indirectly through intervening media, or they may be interconnected between two elements. The specific meanings of the above terms in the invention can be understood by those skilled in the art according to specific situations.

### Embodiment 1

The embodiment discloses a device for replacing the oral tracheal intubation with nasal tracheal intubation, comprising a semicircular tube 1 and a guide tube 2, the side of the semicircular tube 1 being provided with an opening 11 for placement of the guide tube 2 or tracheal intubation, and said opening extend to two ends of the semicircular tube so that the semicircular tube 1 is U-shaped without external force.

The diameter of the guide tube 2 is smaller than that of the semicircular tube 1, and magnetic portions 3 paired with each other are provided on the inner wall of the guide tube 2 as well as on the side wall of the guide tube 2 near the end position. Said magnetic portions 3 are provided on the inner wall of the semicircular tube 1 near the middle position.

Said guide tube 2 is provided with a scale 4 showing the depth of insertion; said guide tube scale 4 is shown as 5-65 cm and said guide tube 2 has a length of 65-75 cm.

The using method of the invention is described below with reference to the accompanying drawings:

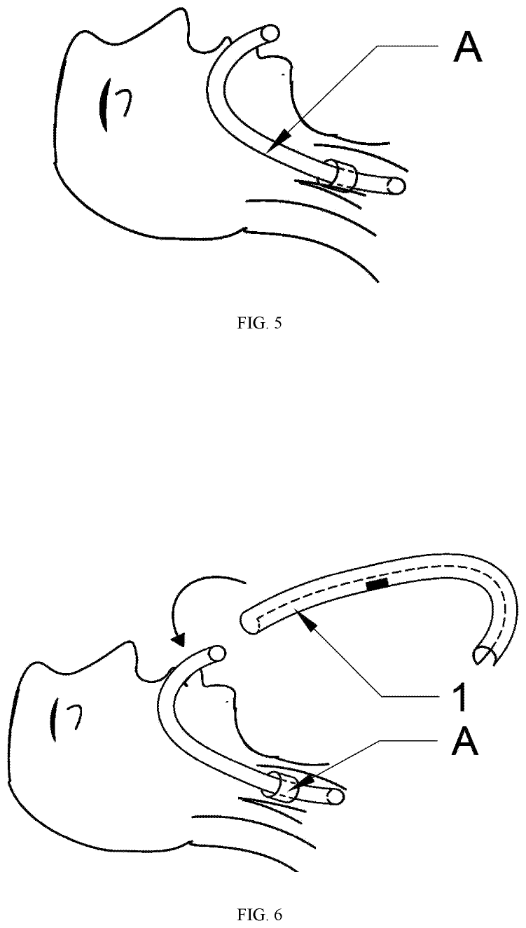

As shown in FIG. 5, the patient is generally intubated via oral tracheal intubation after emergency resuscitation, and when the resuscitation has been completed for a period of time, it is assessed by a physician that the oral tracheal intubation needs to be replaced with nasal tracheal intubation.

The specific operation steps are:

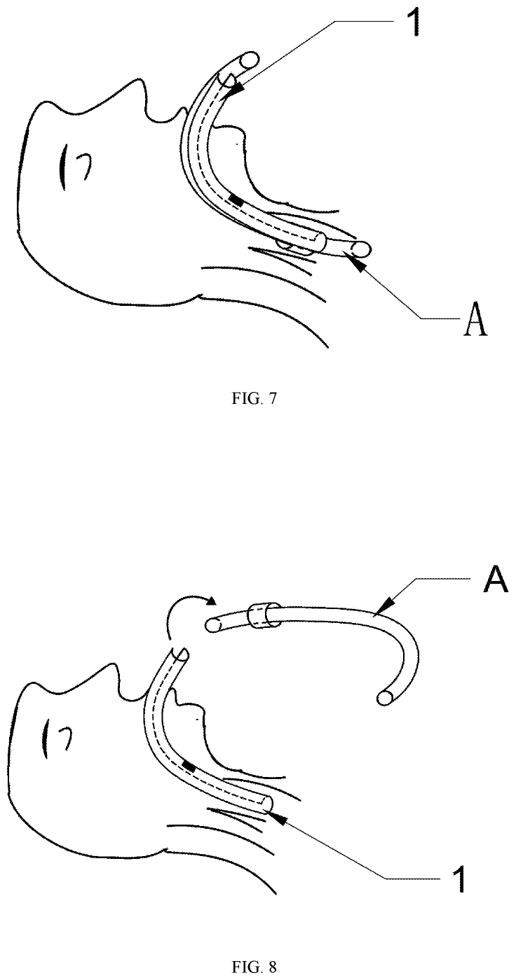

S1. Firstly, the gas in the balloon in the oral tracheal intubation A was extracted, and then the semicircular tube 1 wraps around and fits the outer edge of the oral tracheal intubation A and was gently delivered into the airway as shown in FIG. 6 and FIG. 7. Due to the existence of the oral tracheal intubation A, the semicircular tube 1 can be smoothly delivered into the interior of the airway under the guidance of the oral tracheal intubation.

S2. When the semicircular tube 1 is inserted to a suitable depth (about 20-22 cm), as shown in FIG. 8, the oral tracheal intubation A is withdrawn. At this time, since the semicircular tube 1 has been placed into the airway, the semicircular tube 1 can be used to connect respiratory assistance device to maintain the patient's oxygenation if the condition requires it.

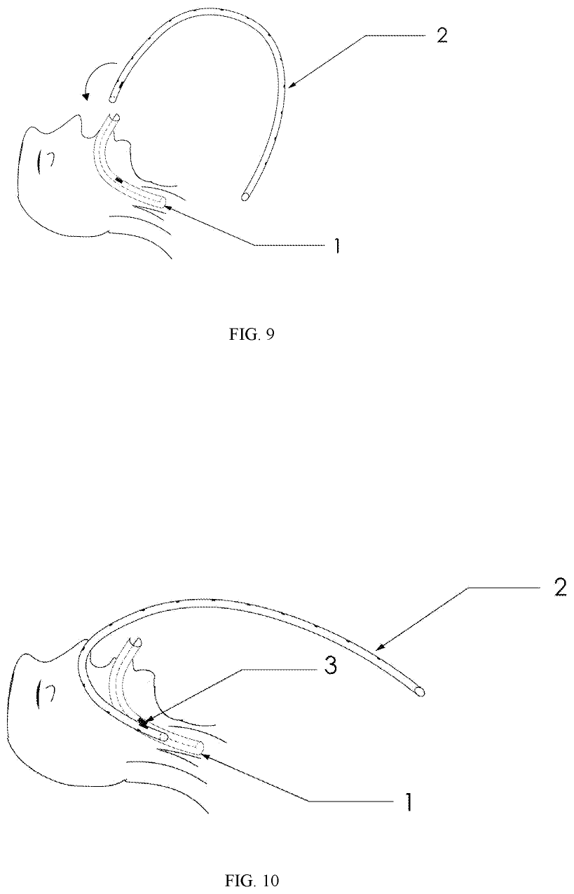

S3. As shown in FIG. 9 and FIG. 10, the guide tube 2 is delivered to the position of the glottis via the nasal passage, at which time the magnetic portions 3 of the semicircular tube 1 and the guide tube 2 are attracted to each other in the vicinity of the glottis, so as to cause the semicircular tube 1 and the guide tube 2 to be combined, and then the direction in which the guide tube 2 is about to be slid into the airway is precisely positioned, a slight force is then applied to slide the guide tube 2 into the airway subsequently. The scale 4 determines whether the guide tube reaches the appropriate anatomical depth, which is about 25 cm for an ordinary adult, wherein 24 cm for women and 26 cm for men.

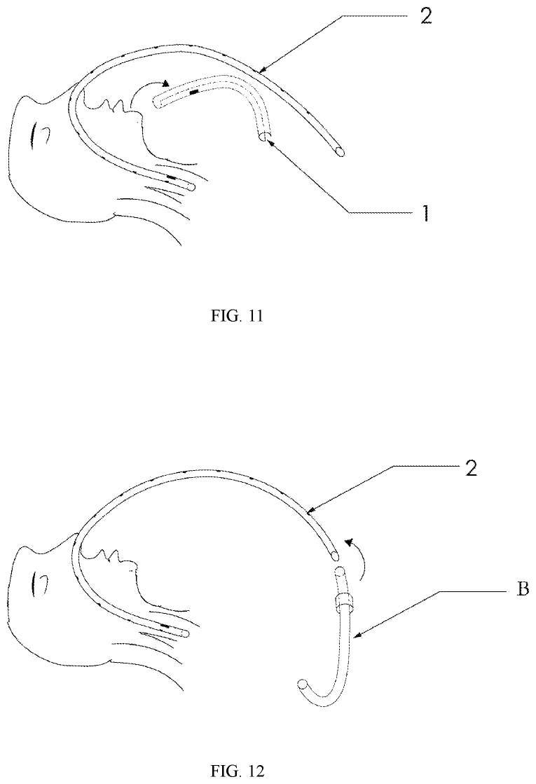

S4. As shown in FIG. 11, the semicircular tube 1 is withdrawn from the oral cavity. due to the existence of opening 11, when withdrawing the semicircular tube 1, it will not affect the position of the guide tube 2, and it will not pull on the guide tube 2 and further irritate the patient's airway.

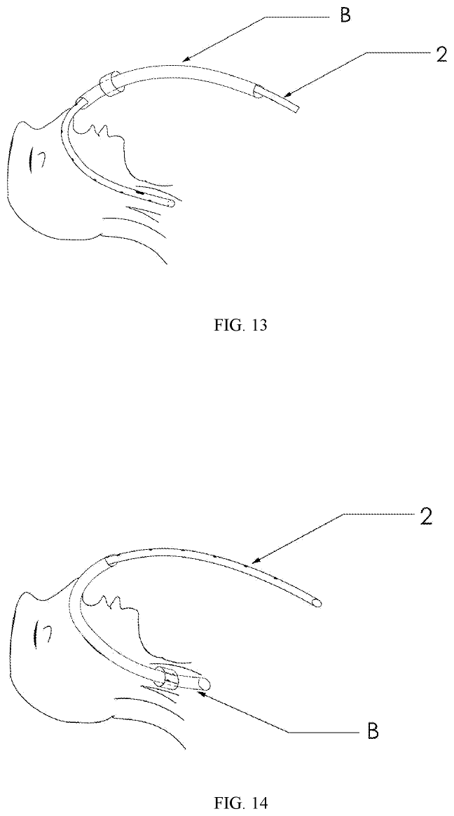

S5. As shown from FIG. 12 to FIG. 14, the nasal tracheal intubation B is fed into the airway along the guide tube 2 to reach a suitable depth of intubation.

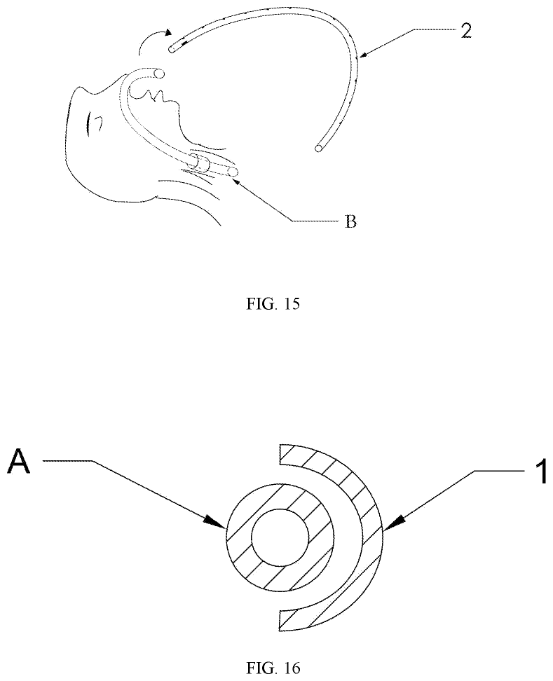

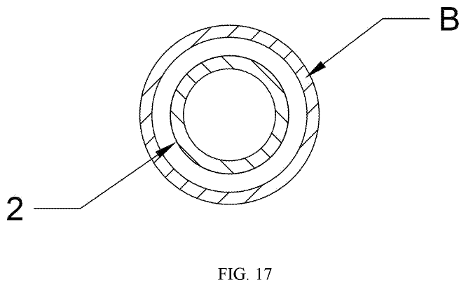

S6. After reconfirming that the nasal tracheal intubation B has been successfully retained by conventional auscultation or testing methods such as End-tidal carbon dioxide, withdraw guide tube 2 (FIG. 15). Follow the subsequent routine steps to fix the nasal tracheal intubation B and connect the respiratory assistance device. At this point, the replacement of oral tracheal intubation with nasal tracheal intubation was completed.

The invention can effectively assist in replacing the oral tracheal intubation with nasal tracheal intubation, reduce the multiple risks that may exist in the process of replacing intubation, improve the patient's respiratory support method, and improve the prognosis.

The invention and its embodiments have been described above, but the description is not limited thereto; only one embodiment of the invention is shown in the drawings, and the actual structure is not limited thereto. In general, it is to be understood by those skilled in the art that non-creative design of structural forms and embodiments that are similar to the technical solutions without departing from the spirit of the invention shall all fall within the protective scope of the invention.

1. A device for replacing the oral tracheal intubation with nasal tracheal intubation, comprising a semicircular tube and a guide tube, the side of the semicircular tube being provided with an opening for placement of the guide tube or tracheal intubation; the diameter of the guide tube is smaller than that of the semicircular tube, and magnetic portions paired with each other are provided on the inner wall of the semicircular tube as well as on the side wall of the guide tube near the end position.

2. The device for replacing the oral tracheal intubation with nasal tracheal intubation of claim 1, wherein said opening extend to two ends of the semicircular tube so that the semicircular tube is U-shaped without external force.

3. The device for replacing the oral tracheal intubation with nasal tracheal intubation of claim 1, wherein said guide tube is provided with a scale showing the depth of insertion.

4. The device for replacing the oral tracheal intubation with nasal tracheal intubation of claim 3, wherein said guide tube scale is shown as 5-65 cm and said guide tube has a length of 65-75 cm.

5. The device for replacing the oral tracheal intubation with nasal tracheal intubation of claim 1, wherein said magnetic portions are provided on the inner wall of the semicircular tube near the middle position.
